(19) United States
(12) Patent Application Publication
(10) Pub. No.: US 2025/0143632 A1
(43) Pub. Date: May 8, 2025

(54) SYSTEM AND METHOD FOR TARGETED MONITORING OF A PATIENT IN A BED FOR PRESSURE INJURY (BED SORE) REDUCTION

(71) Applicant: Covidien LP, Mansfield, MA (US)

(72) Inventors: Rakesh Sethi, Vancouver (CA); Paul S. Addison, Edinburgh (GB); Dean Montgomery, Edinburgh (GB)

(21) Appl. No.: 18/903,364

(22) Filed: Oct. 1, 2024

Related U.S. Application Data
(60) Provisional application No. 63/596,132, filed on Nov. 3, 2023.

(51) Int. Cl. A61B 5/00; G06V 10/764; G06V 20/52; G06V 40/10; G16H 40/67

(52) U.S. Cl. CPC A61B 5/447; A61B 5/0077; A61B 5/7275; A61B 5/7405; A61B 5/742; A61B 5/746; G06V 10/764; G06V 20/52; G06V 40/10; G16H 40/67; G06V 2201/03

(57) ABSTRACT

A computer-implemented method for targeted monitoring of a patient in a bed comprises: receiving an image of the bed; predicting, based on a posture of the patient in the image, a pressure score for one or more contact regions between the patient and the bed; and providing an indication of the pressure score for the one or more contact regions.

### CROSS-REFERENCE TO RELATED APPLICATION

The present application claims the benefit of U.S. Provisional Patent Application Ser. No.: 63/596,132, filed on Nov. 3, 2023, the entire content of which is incorporated herein by reference.

### FIELD

The disclosure relates to a system and method for targeted monitoring of a patient in a bed for pressure injury (bed sore) reduction.

### BACKGROUND

A pressure injury (PI) (also referred to as a bed sore) is localized damage to the skin and underlying soft tissue commonly occurring, for example, during hospital stays or prolonged stays in bed at home. There are many contributing factors, occurring due to a combination of physiological events and external conditions. Tissue ischemia at pressure points as a result of compression of tissue in the bone prominence area due to prolonged external pressure, shearing force, and prolonged contact with hard surfaces have been found to be the main cause of pressure injuries. In addition, to the detrimental effect on patient health, pressure injuries are estimated to cost the US healthcare system an estimated $9.1-$11.6 billion annually. However, it is estimated that 95% are preventable, and, in one study, it was found that nearly 40% of nurses at small-and medium-sized hospitals do not have adequate education or experience related to PI care.

The Applicant's earlier patent application, US2022/0167880, describes a method and system for determining patient posture with a view to determining whether a patient has been in a posture for too long and requires to be moved in order to prevent bed sores from forming. The entire content of US2022/0167880 is incorporated herein by reference.

It is an aim of the present disclosure to provide a system and method for more targeted monitoring of a patient in a bed for improved pressure injury reduction.

### SUMMARY

In general, this disclosure proposes a technique for targeted monitoring of a patient in a bed for improved pressure injury reduction. This technique enables identification of specific areas where bed sores may occur for a given patient and therefore the patient or care provider may be instructed to move a specific body part to reduce the risk of pressure injuries.

According to one aspect of the present disclosure, there is provided a computer-implemented method for targeted monitoring of a patient in a bed comprising:
i. receiving an image of the bed;
ii. predicting, based on a posture of the patient in the image, a pressure score for one or more contact regions between the patient and the bed; and
iii. providing an indication of the pressure score for the one or more contact regions.

Thus, embodiments of this disclosure provide a method for targeted monitoring of a patient in a bed, in which a pressure score for at least one contact region is predicted and indicated to a user (e.g. patient or care provider). Thus, the patient/care provider is informed of specific areas that may be vulnerable to pressure injury and therefore the care of a patient may be improved. In addition, due to the targeted nature of the system, the precious resources of time and staff can be better managed to move only the patients and/or only the parts of each patient that may be most likely to suffer from a pressure injury and unnecessary moving can be avoided.

The step of providing the indication of the pressure score may comprise generating a pressure map based on the pressure score for the one or more contact regions.

The pressure score may relate generally to a prediction of pressure distribution associated with a contact region (e.g. of a particular body part such as an arm/leg/back/chest/side/ankle etc.).

The pressure score may be a quantitative score, for example, based on calculated pressure in an area of concern. In some cases, the pressure score may be based on a predefined scale or classification, for example, using the Braden Scale for Predicting Pressure Ulcer Risk.

The pressure score may be a qualitative score, for example, relating to a low, medium or high risk of developing a pressure injury.

A pressure score of zero may denote no or very little contact or pressure.

The step of predicting the pressure score may comprise detecting, in the image, a patient on the bed; analysing the image to determine a posture of the patient; and inferring one or more contact regions between the patient and the bed. The one or more contact regions may be inferred from the posture of the patient.

The method may comprise receiving an indication of a weight of the patient and using the indication of the weight when predicting the pressure score. The indication of the weight may be an actual weight of the patient or an estimated weight of the patient. In some embodiments, a numerical weight may not be required and instead, the indication of weight may be conveyed by selection of a weight category (e.g. extremely underweight, underweight, average weight, overweight, extremely overweight, etc.).

The method may comprise receiving a three dimensional morphology of the patient and using the three dimensional morphology when predicting the pressure score. The three dimensional morphology may comprise the shape (i.e. stature) of the patient and optionally one or more of their height, weight and other measurements (e.g. waist size, chest size, hip size, neck size, thigh size, leg length, arm length, etc.).

The method may comprise obtaining or creating the three dimensional morphology of the patient using a depth sensing device. The depth sensing device may also be used in detecting the patient in the bed and/or determining the posture of the patient.

The method may comprise receiving patient demographic data and using the patient demographic data when predicting the pressure score. The patient demographic data may comprise one or more of: gender, age, ethnicity and occupation.

The method may further comprise:
iv. receiving one or more further images of the bed;
v. predicting, based on a posture of the patient in the one or more further images, a pressure score for one or more contact regions between the patient and the bed; and
vi. providing an indication of the pressure score for the one or more contact regions.

The method may be applied continuously (e.g. using a video camera to continuously capture images) or at predefined intervals (e.g. capturing successive images).

The method may further comprise determining whether a pressure score based on the posture of the patient in the image or the one or more further images, exceeds a threshold.

The threshold may be based on one or more of:
b. a maximum pressure score (which, in some case may be any non-zero pressure score or a pressure score corresponding to a predetermined e.g. high or dangerously high, pressure); and
c. a time in which a same contact region is determined to be in contact between a first image and a later image.

In some cases a combination of time and pressure may be considered, for example, if a medium pressure is exerted for an extended time period, a risk of pressure injury may increase.

The method may further comprise issuing a notification when the pressure score exceeds the threshold.

The notification may comprise one or more of:
i. sounding an alarm or warning message;
ii. turning on a light; and
iii. advising the patient or care provider to adjust the posture of the patient at least in relation to the one or more contact regions.

The warning message may be audible, visual or both.

The advising of the patient or care provider may be carried out by sending an electronic signal to a remote control device or application used by the patient or care provider.

The method may further comprise receiving patient physiological data and/or patient clinical data and using the patient physiological data and/or patient clinical data to determine the threshold.

The patient physiological data may comprise one or more of: i) respiratory rate, ii) oxygen saturation, iii) temperature, iv) systolic blood pressure, v) pulse rate and vi) level of consciousness.

The patient clinical data may comprise one or more of: i) patient wellbeing, ii) patient disease/injury, iii) treatment, iv) medication, v) risk factors (e.g. smoker, alcohol intake).

The method may comprise receiving the image as a still image or extracting the image from a video stream.

The method may comprise applying a classification procedure to classify the pressure score as representative or at least indicative of a low pressure, a medium pressure or a high pressure.

The step of predicting the pressure score and/or a step of detecting the patient and/or a step of analysing the image may be carried out by one or more trained machine learning models.

The trained machine learning model(s) may comprise one or more of: a decision tree, a k-nearest neighbors (kNN) algorithm, an Adaptive Boosting (AdaBoost) technique, a Random Forest algorithm, a Neural Network, and a Support Vector Machine (SVM), a deep learning model, a convolutional neural network, a recurrent neural network, or a transformer model.

The machine learning model may be trained using a suitable data set in a conventional manner.

The steps of receiving an image of the bed; detecting, in the image, a patient on the bed; and analysing the image to determine a posture of the patient; may be carried out using any suitable method, such as the method described in US2022/0167880.

According to a second aspect of this disclosure, there is provided a system for targeted monitoring of a patient in a bed comprising:
d. a depth sensing device configured to obtain an image of a bed;
e. a processing resource configured to:
i. receive the image of the bed;
ii. predict, based on a posture of the patient in the image, a pressure score for one or more contact regions between the patient and the bed; and
iii. provide an indication of the pressure score for the one or more contact regions.

The depth sensing device may comprise at least one of: a depth sensing camera, a stereo camera, a camera cluster, a camera array, and a motion sensor.

The system may further comprise a display configured to provide the indication of the pressure score for the one or more contact regions visually to a user.

The system may further comprise a memory for storing one or more of: a posture of the patient; and a predicted pressure score for one or more contact regions. Each posture and/or pressure score may be time-stamped based on a time when the corresponding image was captured.

The system may be further configured to flag a change in posture and/or pressure score for a particular contact region. The change may occur between successive images. A timestamp and optionally details associated with the change may be stored in the memory.

According to a third aspect of this disclosure, there is provided a non-transitory machine-readable medium having instructions recorded thereon for execution by a processor to:
iv. receive an image of a bed;
v. predict, based on a posture of a patient in the image, a pressure score for one or more contact regions between the patient and the bed; and
vi. provide an indication of the pressure score for the one or more contact regions.

### BRIEF DESCRIPTION OF THE PREFERRED EMBODIMENTS

Many aspects of the present disclosure can be better understood with reference to the following drawings. The components in the drawing are not necessarily to scale. Instead, emphasis is placed on illustrating clearly the principles of the present disclosure. The drawings should not be taken to limit the disclosure to the specific embodiments depicted but are for explanation and understanding only.

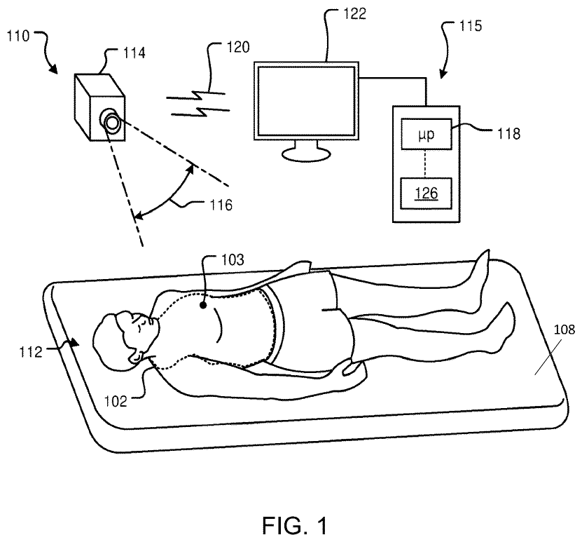

FIG. 1 shows a schematic view of a system for targeted monitoring of a patient in a bed in accordance with the present disclosure.

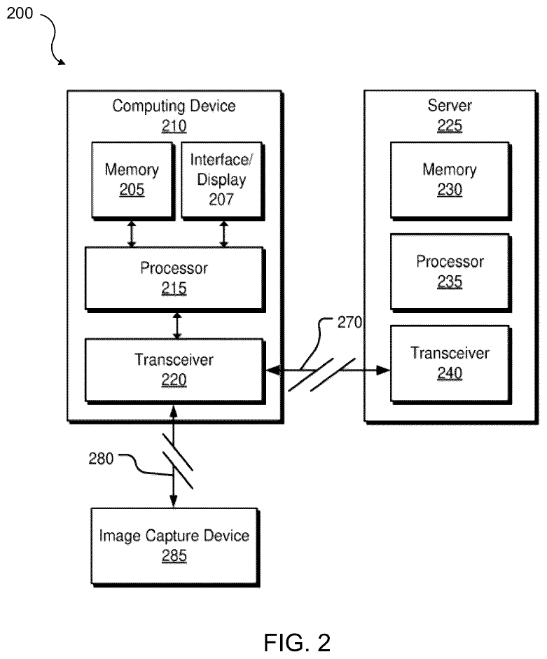

FIG. 2 shows a block diagram of a system for targeted monitoring of a patient in a bed, including a computing device, a server and an image capturing device.

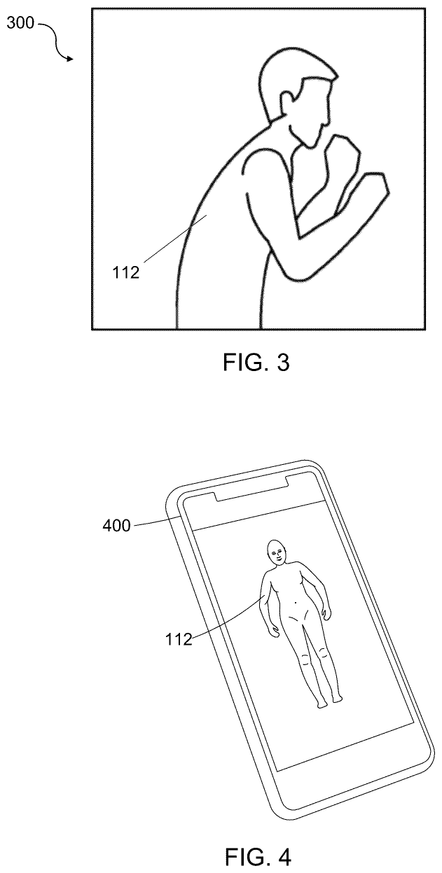

FIG. 3 shows a schematic view of an image of a patient in a bed.

FIG. 4 shows a three dimensional morphology of a patient obtained using a three dimensional scanner and viewed on a mobile device.

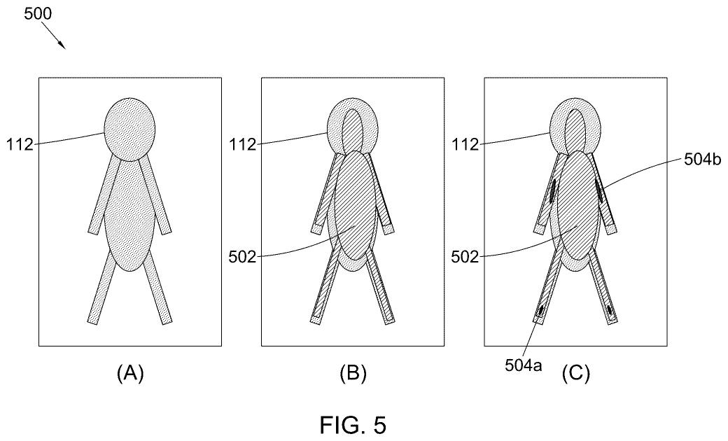

FIG. 5 shows a series of schematic illustrations of a patient on a bed where (A) shows a posture of the patient, (B) shows contact regions between the patient and bed, and (C) shows predicted high pressure areas in the contact regions.

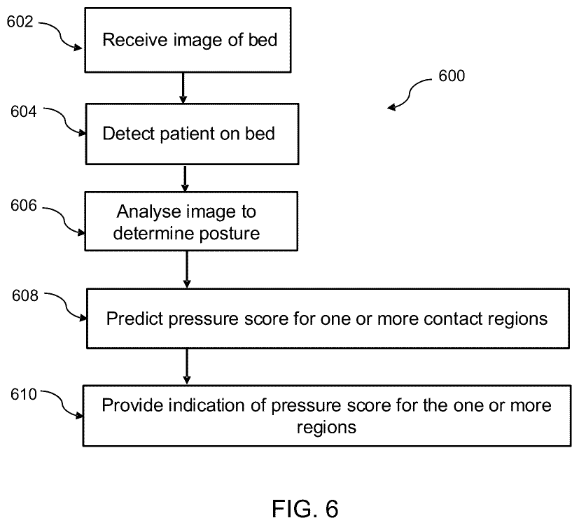

FIG. 6 shows a flow chart illustrating a method for targeted monitoring of a patient in a bed in accordance with the present disclosure.

### DETAILED DESCRIPTION OF THE PREFERRED EMBODIMENTS

Specific details of several embodiment of the present technology are described herein with reference to the accompany figures. Although many of the embodiments are described with respect to devices, systems, and methods for video-based monitoring of a human patient's position when lying in bed, other applications and other embodiments in addition to those described herein are within the scope of the present technology. For example, at least some embodiments of the present technology can be useful for monitoring of non-patients (e.g., elderly or infirm individuals within their homes). It should be noted that other embodiments in addition to those disclosed herein are within the scope of the present technology. Further, embodiments of the present technology can have different configurations, components, and/or procedures than those shown or described herein. Moreover, a person of ordinary skill in the art will understand that embodiments of the present technology can have configurations, components, and/or procedures in addition to those shown or described herein and that these and other embodiments can be without several of the configurations, components, and/or procedures shown or described herein without deviating from the present technology.

Generally speaking, the disclosure provides systems and methods for determining regions where bed sores may develop for an individual, monitoring them in real time and targeting specific regions for care, while optimizing staff resources to reduce the risk of pressure injuries.

The system may be based on that described in US2022/0167880 for detecting, in an image, a patient on the bed and analysing the image to determine a posture of the patient.

FIG. 1 shows a schematic view of a system 110 for targeted monitoring of a patient 112 in a bed 108 in accordance with the present disclosure. The system 110 includes a non-contact detector 114 and a computing device 115. In some embodiments, the detector 114 can include one or more image capture devices, such as one or more video cameras. In the illustrated embodiment, the non-contact detector 114 is in the form of a video camera. However, in other embodiments, the non-contact detector 114 may be in the form of a still image camera. The non-contact detector 114 of the system 110 is placed remote from the patient 112. More specifically, the video camera of the non-contact detector 114 is positioned remote from the patient 112 in that it is spaced apart from and does not contact the patient 112. The non-contact detector 114 includes a detector exposed to a field of view (FOV) 116 that encompasses at least a portion of the patient 112 on the bed 108.

The non-contact detector 114 can capture a sequence of images over time. The non-contact detector 114 can be a depth sensing camera, such as a Kinect camera from Microsoft Corp. (Redmond, Washington) or Intel camera such as the D415, D435, and SR305 cameras from Intel Corp, (Santa Clara, California). A depth sensing camera can detect a distance between the camera and objects within its field of view. Such information can be used to determine that a patient 112 is within the FOV 116 of the detector 114 and/or to determine one or more regions of interest (ROI) 102 to monitor on the patient 112. Once a ROI is identified, the ROI can be monitored over time, and the changes in depth of regions (e.g., pixels) within the ROI 102 can represent movements of the patient 112. While a depth sending camera is described previously, it should be appreciated that other types of cameras using other imaging modalities (e.g., RGB, thermal, IR, etc.) can additionally or alternatively be used in the systems and methods described herein.

In some embodiments, the system 110 determines a skeleton-like outline of the patient 112 to identify a point or points from which to extrapolate a ROI 102. For example, a skeleton-like outline can be used to find a center point of a chest, shoulder points, waist points, and/or any other points on a body of the patient 112. These points can be used to determine one or more ROIs 102. For example, a ROI 102 can be defined by filling in area around a center point 103 of the chest, as shown in FIG. 1. Certain determined points can define an outer edge of the ROI 102, such as shoulder points. In other embodiments, instead of using a skeleton, other points may be used to establish a ROI 102. For example, a face can be recognized, and a chest area inferred in proportion and spatial relation to the face. In other embodiments, a reference point of a patient's chest can be obtained (e.g., through a previous 3-D scan of the patient), and the reference point can be registered with a current 3-D scan of the patient. In these and other embodiments, the system 110 can define a ROI around a point using parts of the patient 112 that are within a range of depths from the detector 114. In other words, once the system 110 determines a point from which to extrapolate a ROI, the system 110 can utilize depth information from the depth sensing camera to fill out the ROI. For example, if the point 103 on the chest is selected, parts of the patient 112 around the point 103 that are a similar depth from the detector 114 as the point 103 are used to determine the ROI 102.

In another example, the patient 112 can wear specially configured clothing (not shown) that includes one or more features to indicate points on the body of the patient 112, such as the patient's shoulders and/or the center of the patient's chest. The one or more features can include a visually encoded message (e.g., bar code, QR code, etc.), and/or brightly colored shapes that contrast with the rest of the patient's clothing. In these and other embodiments, the one or more features can include one or more sensors that are configured to indicate their positions by transmitting light or other information to the camera 114. In these and still other embodiments, the one or more features can include a grid or another identifiable pattern to aid the system 110 in recognizing the patient 112 and/or the patient's movement. In some embodiments, the one or more features can be stuck on the clothing using a fastening mechanism such as adhesive, a pin, etc. For example, a small sticker can be placed on a patient's shoulders and/or on the center of the patient's chest that can be easily identified within an image captured by the camera 114. The system 110 can recognize the one or more features on the patient's clothing to identify specific points on the body of the patient 112. In turn, the system 100 can use these points to recognize the patient 112 and/or to define a ROI.

In some embodiments, the system 100 can receive user input to identify a starting point for defining a ROI. For example, an image can be reproduced on a display 122 of the system 110, allowing a user of the system 110 to select a patient 112 for monitoring (which can be helpful where multiple objects are within the FOV 116 of the camera 114) and/or allowing the user to select a point on the patient 112 from which a ROI can be determined (such as the point 103 on the chest of the patient 112). In other embodiments, other methods for identifying a patient 112, identifying points on the patient 112, and/or defining one or more ROI's can be used.

The images detected by the camera 114 can be sent to the computing device 115 through a wired or wireless connection 120. The computing device 115 can include a processor 118 (e.g. a microprocessor), the display 122, and/or hardware memory 126 for storing software and computer instructions. Sequential image frames of the patient 112 may be recorded by the video camera 114 and sent to the processor 118 for analysis. The display 122 can be remote from the camera 114, such as a video screen positioned separately from the processor 118 and the memory 126. Other embodiments of the computing device 115 can have different, fewer, or additional components than shown in FIG. 1. In some embodiments, the computing device 115 can be a server. In other embodiments, the computing device 115 of FIG. 1 can be additionally connected to a server (e.g., as shown in FIG. 2 and discussed in greater detail below). The captured images/video can be processed or analyzed at the computing device 115 and/or a server to determine, e.g., a patient's position while lying in bed or a patient's change from a first position to second position while lying in bed. In some embodiments, some or all of the processing may be performed by the camera, such as by a processor integrated into the camera or when some or all of the computing device 115 is incorporated into the camera.

FIG. 2 is a block diagram illustrating a video-based patient monitoring system 200 (e.g., the video-based patient monitoring system 110 shown in FIG. 1) having a computing device 210, a server 225, and one or more image capture devices 285, and configured in accordance with various embodiments of the present technology. In various embodiments, fewer, additional, and/or different components can be used in the system 200. The computing device 210 includes a processor 215 that is coupled to a memory 205. The processor 215 can store and recall data and applications in the memory 205, including applications that process information and send commands/signals according to any of the methods disclosed herein. The processor 215 can also (i) display objects, applications, data, etc. on an interface/display 207 and/or (ii) receive inputs through the interface/display 207. As shown, the processor 215 is also coupled to a transceiver 220.

The computing device 210 can communicate with other devices, such as the server 225 and/or the image capture device(s) 285 via (e.g., wired or wireless) connections 270 and/or 280, respectively. For example, the computing device 210 can send to the server 225 information determined about a patient from images captured by the image capture device(s) 285. The computing device 210 can be the computing device 115 of FIG. 1. Accordingly, the computing device 210 can be located remotely from the image capture device(s) 285, or it can be local and close to the image capture device(s) 285 (e.g., in the same room). In various embodiments disclosed herein, the processor 215 of the computing device 210 can perform the steps disclosed herein. In other embodiments, the steps can be performed on a processor 235 of the server 225. In some embodiments, the various steps and methods disclosed herein can be performed by both of the processors 215 and 235. In some embodiments, certain steps can be performed by the processor 215 while others are performed by the processor 235. In some embodiments, information determined by the processor 215 can be sent to the server 225 for storage and/or further processing.

In some embodiments, the image capture device(s) 285 are remote sensing device(s), such as depth sensing video camera(s), as described above with respect to FIG. 1. In some embodiments, the image capture device(s) 285 can be or include some other type(s) of device(s), such as proximity sensors or proximity sensor arrays, heat or infrared sensors/cameras, sound/acoustic or radio wave emitters/detectors, or other devices that include a field of view and can be used to monitor the location and/or characteristics of a patient or a region of interest (ROI) on the patient. Body imaging technology can also be utilized according to the methods disclosed herein. For example, backscatter x-ray or millimeter wave scanning technology can be utilized to scan a patient, which can be used to define and/or monitor a ROI. Advantageously, such technologies can be able to “see” through clothing, bedding, or other materials while giving an accurate representation of the patient's skin. This can allow for more accurate measurements, particularly if the patient is wearing baggy clothing or is under bedding. The image capture device(s) 285 can be described as local because they are relatively close in proximity to a patient such that at least a part of a patient is within the field of view of the image capture device(s) 285. In some embodiments, the image capture device(s) 285 can be adjustable to ensure that the patient is captured in the field of view. For example, the image capture device(s) 285 can be physically movable, can have a changeable orientation (such as by rotating or panning), and/or can be capable of changing a focus, zoom, or other characteristic to allow the image capture device(s) 285 to adequately capture images of a patient and/or a ROI of the patient. In various embodiments, for example, the image capture device(s) 285 can focus on a ROI, zoom in on the ROI, center the ROI within a field of view by moving the image capture device(s) 285, or otherwise adjust the field of view to allow for better and/or more accurate tracking/measurement of the ROI.

The server 225 includes a processor 235 that is coupled to a memory 230. The processor 235 can store and recall data and applications in the memory 230. The processor 235 is also coupled to a transceiver 240. In some embodiments, the processor 235, and consequently the server 225, can communicate with other devices, such as the computing device 210 through the connection 270.

The devices shown in the illustrative embodiment can be utilized in various ways. For example, either the connections 270 or 280 can be varied. One or both of the connections 270 and 280 can be a hard-wired connection. A hard-wired connection can involve connecting the devices through a USB (universal serial bus) port, serial port, parallel port, or other type of wired connection that can facilitate the transfer of data and information between a processor of a device and a second processor of a second device. In another embodiment, either of the connections 270 and 280 can be a dock where one device can plug into another device. In other embodiments, either of the connections 270 and 280 can be a wireless connection. These connections can take the form of any sort of wireless connection, including, but not limited to, Bluetooth connectivity, Wi-Fi connectivity, infrared, visible light, radio frequency (RF) signals, or other wireless protocols/methods. For example, other possible modes of wireless communication can include near-field communications, such as passive radio-frequency identification (RFID) and active RFID technologies. RFID and similar near-field communications can allow the various devices to communicate in short range when they are placed proximate to one another. In yet another embodiment, the various devices can connect through an internet (or other network) connection. That is, either of the connections 270 and 280 can represent several different computing devices and network components that allow the various devices to communicate through the internet, either through a hard-wired or wireless connection. Either of the connections 270 and 280 can be formed of a combination of several modes of connection.

The configuration of the devices in FIG. 2 is merely one physical system 200 on which the disclosed embodiments can be executed. Other configurations of the devices shown can exist to practice the disclosed embodiments. Further, configurations of additional or fewer devices than the devices shown in FIG. 2 can exist to practice the disclosed embodiments. Additionally, the devices shown in FIG. 2 can be combined to allow for fewer devices than shown or can be separated such that more than the three devices exist in a system. It will be appreciated that many various combinations of computing devices can execute the methods and systems disclosed herein. Examples of such computing devices can include other types of medical devices and sensors, infrared cameras/detectors, night vision cameras/detectors, other types of cameras, augmented reality goggles, virtual reality goggles, mixed reality goggle, radio frequency transmitters/receivers, smart phones, personal computers, servers, laptop computers, tablets, blackberries, RFID enabled devices, smart watch or wearables, or any combinations of such devices.

Further details of how the system of FIGS. 1 and 2 may be used to determine the posture of the patient 112 in the bed 118 are provided in US2022/0167880.

In some embodiments, the system may be based on a machine learning model trained on a large dataset of patient images in various postures. These may include, for example, images classified as left, right, prone, supine, sitting, and “no patient”.

FIG. 3 shows a schematic view of an image 300 of a patient 112 lying on their side in a bed. The image 300 of FIG. 3 would be labelled ‘left’, as the patient is lying on their left-hand side.

In addition to the image 300, patient demographic data including for example, height, weight, gender, age, ethnicity and occupation may be input to the system 110. In some embodiments, a three dimensional morphology of the patient 112 may be created or obtained for use in a method according to the present disclosure.

FIG. 4 shows an example three dimensional morphology of a patient 112, as viewed on a mobile device 400. This three dimensional morphology has been obtained using a commercial system that maps patient body morphology from a three dimensional depth camera scan. Thus, the three dimensional morphology may be constructed from views of the patient 112 taken using the depth camera 114 described above. In some cases, an initial scanning step may be required prior to the patient lying on the bed. However, in other cases a three dimensional morphology of the patient 112 may be created through prolonged monitoring of the patient 112 in the bed 108 (e.g. as the patient rolls into different positions, or as they enter/exit the bed, different views of the patient can be taken using the depth camera 114 and then used to construct a three dimensional map of the patient's body, without requiring an explicit scanning step). In some embodiments, a dedicated three dimensional scanner (e.g. a Proscanner from Fit3D or a Portal MX scanner from Texel) may be employed.

In embodiments of the disclosure, the system will predict, based on the posture of the patient 112 in the image 300, a pressure score for one or more contact regions between the patient 112 and the bed 108.

In some cases, knowledge of the patient's body morphology may first be used to infer contact regions of the patient 112 with the bed 108 (e.g. mattress). The patient's weight may then be used to infer pressure across the contact regions with the bed.

FIG. 5 shows a series of schematic illustrations of a patient 112 on a bed 108 where FIG. 5(A) shows a posture of the patient 112, FIG. 5(B) shows contact regions 502 between the patient 112 and bed 108, and FIG. 5(C) shows predicted high pressure areas 504a, 504b in the contact regions 502.

More specifically, FIG. 5(A) shows an outline of a patient 112 lying supine on a mattress. The hatched region of FIG. 5(B) shows the inferred contact region 502 between the patient 112 and the mattress. This inference may be determined from a machine learning model, which has learned from training data. The training data may comprise depth information obtained from the depth camera 114 and further data obtained, for example, by using pressure probes to determine contact regions (and pressure) between the patient 112 and the bed 108.

For example, the machine learning model may comprise a multilayer deep neural network. The network may be developed through transfer learning of existing neural networks, such as Alexnet, ResNet, GoogLeNet, ViT, etc. Alternatively, the machine learning model may be developed from scratch. The machine learning model may include layers such as an input layer, a convolutional layer, a relu layer, pooling layers, dropout layers, output layers, a softmax layer, etc or any suitable combination of such layers. Any suitable machine learning model and/or deep learning model may be used, for example but not limited to, a neural network, a convolutional neural network, a recurrent neural network, a transformer model, a decision tree, a k-nearest neighbors (kNN) algorithm, an Adaptive Boosting (AdaBoost) technique, a Random Forest algorithm, or a Support Vector Machine (SVM).

The model may be trained using any suitable training technique in various embodiments, for example using supervised, semi-supervised or unsupervised training techniques.

In some embodiments the training data, which may comprise ground truth data, comprises a series of data sets obtained for a plurality of patients. Each data set in some embodiments comprises a depth image (e.g. a set of pixel values, each pixel value providing a depth value for a corresponding position in a field of view) obtained for a patient at a point in time. The data sets may also include one or more of patient demographic data, patient physiological data and/or patient clinical data. Each training data set in these embodiments may also include pressure data obtained at the respective point in time using pressure sensors positioned beneath the patient. The pressure data comprises pressure as a function of position.

Any suitable pressure sensor may be used, for example an array of capacitive, optical, piezoelectric, magnetic or other sensors. The sensors may be provided in an array for example in a mat or within a mattress. Any suitable known pressure sensor array for use in a clinical setting, for example intended for use on hospital beds may be used in some embodiments.

During the training phase, in some embodiments, the depth image data and optionally the patient demographic data, patient physiological data and/or patient clinical data are provided as inputs to an input layer of the neural network.

In accordance with known techniques the neural network generates weights that propagate through successive layers of the neural network and at an output layer, the neural network outputs a set of values that are predicted pressure values as a function of position or contact region. A loss function or any other suitable process can be used to determine how well the predicted pressure values or contact regions match the ground truth value (e.g. the measured value of pressure or contact region as a function of position). A numerical measure of that match, for example a value of loss parameter is then provided as an input to the machine learning model and the process is repeated. The process is performed for all of the training data sets until the model is trained.

In some embodiments, a U-Net architecture may be employed to output a matrix of pressure values or an image representing the pressure values (e.g. as a pressure map), derived from the inputs. Any other architecture that may be configured to work in a similar way could be employed. For example, a U-Net architecture without skip connections could be used.

Once trained, the machine learning model is able to take a new data set, for example comprising a set of pixel values of depth data, and optionally comprising the patient demographic data, patient physiological data and/or patient clinical data, and provide as an output the corresponding pressure values experienced by the patient as a function of position and/or the predicted contact regions, for example as would be measured by a pressure sensor array if present.

In some embodiments the determined pressure values may be used as a pressure score directly. In other embodiments, the pressure score may be subsequently determined from the pressure values, for example by applying an algorithm to classify each pressure value as being high, medium or low, or being on a desired numerical scale of scores (for example, 1 to 10 or 1 to 100). In some embodiments, the score may represent a more complex or alternative calculation or classification, for example the score may depend on a value of pressure and/or area or proportion of the patient subject to the pressure and/or duration of the pressure. In some embodiments, the pressure score may be calculated as the pressure value multiplied by the area of the patient subject to the pressure and further multiplied by the duration of the applied pressure. This would take into account how long and how much of a patient's skin is being subject to pressure (e.g. high or moderate pressure). A threshold could then be set for this cumulative score.

As described above, the output of the machine learning model may be a set of pressure values. These may be used as the pressure score or may be used in subsequent calculation of the pressure score. In some embodiments the machine learning model may provide the pressure score directly as its output. In such embodiments, a determined pressure score may be included in the ground truth of the training data sets. In various embodiments clinician or other expert input and/or a subjective assessment of discomfort by patients may also be provided as part of the ground truth in the training data. For example, based on pressure measurements and/or inspection of a patient and/or clinical notes, a clinician may provide a score or indication of risk of pressure sore problems and that may, for example, be included in the ground truth as part of the training process.

In some embodiments a separate pre-processing step may be performed, for example to detect, in the image data, a patient on the bed, and to analyse the image to determine a posture of the patient, for instance as described herein. The results of such detection and analysis may then be provided as input to the or a machine learning model (and may be included in the training data sets). In other embodiments, the image data is provided as an input to the machine learning model and the machine learning process then effectively detects the patient, determines the posture, and outputs the score.

In some embodiments more than one machine learning model may be used. For example a first machine learning model may be used to determine posture, for instance using techniques described in US2022/0167880, and then data representing the determined posture may be provided as input to a further machine learning model to determine pressure value and/or pressure score. Alternatively a single machine learning model may be used to determine pressure values and/or score from image data as input, thus effectively combining determination of posture and score using a single machine learning model.

The neural network and the training process has been described above in overview. It will be understood that any suitable number and combination of layers and nodes, in accordance with known techniques, may be used in other embodiments. Similarly, for non-neural network machine learning models any suitable known training techniques specific to such models may be used.

The model may be trained to determine the contact pressures (or pressure scores) across substantially the entire contact region 502 of FIG. 5(B). However, only pressures exceeding a predetermined threshold are indicated in FIG. 5(C) as critical areas 504a and 504b. Thus, only pressures deemed to be beyond a safe limit may be highlighted in FIG. 5(C).

The threshold may be determined based on a critical (e.g. maximum) pressure beyond which a risk of bed sores is likely. Additionally or alternatively, the threshold may be meet when the pressure is above a certain value for a prolonged period of time. In some cases the threshold may be based on a combination of one or more pressures and/or times, for example, in relation to substantially the same contact area.

In some cases, the threshold may be determined based on patient physiological data and/or patient clinical data. For example, the patient physiological data may comprise one or more of: i) respiratory rate, ii) oxygen saturation, iii) temperature, iv) systolic blood pressure, v) pulse rate and vi) level of consciousness and the patient clinical data may comprise one or more of: i) patient wellbeing, ii) patient disease/injury, iii) treatment, iv) medication, v) risk factors (e.g. smoker, alcohol intake). For example, factors such as poor circulation or low blood pressure may increase a risk of pressure injury. Such data may therefore be included in the training data used to train the machine learning model to take into these factors.

The system 110 may connect to electronic records in a hospital and automatically determine, from the patient's records, relevant factors—for example, the system 110 may determine that the patient 112 had surgery on their left side and may therefore lower a time-based threshold for the patient's left side—i.e. ensuring that an alarm will be raised more quickly if the patient is lying on their left side.

The system 110 may be trained for patients 112 with and without bed coverings of various types, bed clothes of various types, varying pillow configurations, etc.

The system 110 may also have the bed 108 shape or position input. If, for example, an upper portion of the bed 108 is in a raised position, this may cause additional pressure on the buttocks and less on the back. This information may therefore be used in making an accurate prediction of the pressure distribution across the patient's body.

The system 110 may be deployed in a patient population where it can learn over time the optimal thresholds to reduce bed sore development. For example, it may learn that for certain patient morphologies and weights that the threshold pressure (or time, or pressure and time) may differ from patients with other morphologies and weights.

By targeting individual patient needs, the system can optimize care and reduce precious staff resources moving patients who are not prone to bed sores.

The system 110 may localize care to a specific body region. For example, if the system detects that a critical region (e.g. 504a or 504b in FIG. 5(C)) needs attention, it could specify only moving or manipulating that region. For example the back of the foot may be identified as a critical region and the carer instructed to manipulate the foot region only to reduce the pressure at that specific point—for example, by placing a pillow under the calf muscle.

The system 110 may provide feedback directly to the patient 112, for example, by an indication on the screen 122, or an audible alert asking the patient 112 to roll over or adjust their position. If the patient 112 does not respond the alert could then be escalated to a relevant member of the care team.

In an alternate embodiment, the system 110 could be used for at-home monitoring, perhaps after discharge from the hospital. In this case, the system 110 could monitor the position of a person in bed 108 overnight (or during the day) and send a report to the person and to clinical staff. The report may include automated suggestions, for example “try sleeping on your right side tonight”.

FIG. 6 shows a flow chart illustrating a method 600 for targeted monitoring of a patient in a bed in accordance with the present disclosure.

The method 600 is a computer-implemented method, which may be carried out by the system of FIG. 1 and FIG. 2. The method 600 comprises a step 602 of receiving an image 300 of the bed 108 and a step 604 of detecting, in the image 300, a patient 112 on the bed 108. A step 606 comprises analysing the image 300 to determine a posture of the patient 112. A step 608 comprises predicting, based on the posture of the patient 112 in the image 300, a pressure score for one or more contact regions 504a, 504b between the patient 122 and the bed 108. A step 610 comprises providing an indication of the pressure score for the one or more contact regions 504a, 504b.

In some embodiments, the step 604 of detecting, in the image 300, a patient 112 on the bed 108 and/or the step 606 of analysing the image 300 to determine a posture of the patient 112 may not be discrete steps. For example, raw data relating to the image 300 could simply be input to a trained machine learning model to predict and output the pressure scores.

For example, the indication may be provided in the form a pressure map illustration similar to that of FIG. 5(C). Additionally or alternatively, the indication may take the form an audible and/or visual alarm or an audible and/or visual warning message advising the patient or care provider to adjust the posture of the patient 112 at least in relation to the one or more contact regions 504a, 504b.

It will be understood that the method 600 may be applied continuously (e.g. using a video camera to continuously capture images) or at predefined intervals (e.g. capturing successive images) so as to monitor a patient 112 over time.

Embodiments of the present disclosure can be employed in many different settings including in hospitals, care homes and in patient homes.

By targeting individual patient needs, the system can optimize care: enhancing care for those who need it and reducing precious staff resources moving patients who (or body parts that) are not critical.

The skilled person will understand that in the preceding description and appended claims, positional terms such as ‘above’, ‘along’, ‘side’, etc. are made with reference to conceptual illustrations, such as those shown in the appended drawings. These terms are used for ease of reference but are not intended to be of limiting nature. These terms are therefore to be understood as referring to an object when in an orientation as shown in the accompanying drawings.

Although the disclosure has been described in terms of preferred embodiments as set forth above, it should be understood that these embodiments are illustrative only and that the claims are not limited to those embodiments. Those skilled in the art will be able to make modifications and alternatives in view of the disclosure, which are contemplated as falling within the scope of the appended claims. Each feature disclosed or illustrated in the present specification may be incorporated in any embodiments, whether alone or in any appropriate combination with any other feature disclosed or illustrated herein.

What is claimed is:

1. A computer-implemented method for targeted monitoring of a patient in a bed comprising:
receiving an image of the bed;
predicting, based on a posture of the patient in the image, a pressure score for one or more contact regions between the patient and the bed; and
providing an indication of the pressure score for the one or more contact regions.

2. The method of claim 1, wherein the step of providing the indication of the pressure score further comprises generating a pressure map based on the pressure score for the one or more contact regions.

3. The method of claim 1, wherein the step of predicting the pressure score further comprises:
detecting, in the image, the patient on the bed;
analyzing the image to determine the posture of the patient; and
inferring one or more contact regions between the patient and the bed.

4. The method of claim 1, further comprising:
receiving an indication of a weight of the patient; and
using the indication of the weight when predicting the pressure score.

5. The method of claim 1, further comprising:
receiving a three dimensional morphology of the patient; and
using the three dimensional morphology when predicting the pressure score.

6. The method of claim 5, further comprising one of obtaining or creating the three dimensional morphology of the patient using a depth sensing device.

7. The method of claim 1, further comprising:
receiving patient demographic data; and
using the patient demographic data when predicting the pressure score.

8. The method of claim 1, further comprising:
receiving one or more further images of the bed;
predicting, based on a further posture of the patient in the one or more further images, the pressure score for the one or more contact regions between the patient and the bed; and
providing the indication of the pressure score for the one or more contact regions.

9. The method of claim 8, further comprising determining whether the pressure score based on the posture of the patient in the image or the further posture of the patient in the one or more further images, exceeds a threshold.

10. The method of claim 9, wherein the threshold is based on one or more of:
a maximum pressure score; and
a time in which a same contact region is determined to be in contact between a first image and a later image.

11. The method of claim 9, further comprising issuing a notification when the pressure score exceeds the threshold.

12. The method of claim 11, wherein the notification comprises one or more of:
sounding an alarm or warning message;
turning on a light; and
advising the patient or a care provider to adjust the posture of the patient at least in relation to the one or more contact regions.

13. The method of claim 9, further comprising receiving at least one of patient physiological data or patient clinical data and using the patient physiological data or patient clinical data to determine the threshold.

14. The method of claim 1, further comprising one of receiving the image as a still image or extracting the image from a video stream.

15. The method of claim 1, further comprising applying a classification procedure to classify the pressure score as one of representative of or indicative of a low pressure, a medium pressure or a high pressure.

16. The method of claim 1, wherein at least a step of predicting the pressure score, detecting the patient, or analyzing the image is carried out by one or more trained machine learning models.

17. The method of claim 16, wherein the one or more trained machine learning models comprises one or more of: a decision tree, a k-nearest neighbors (kNN) algorithm, an Adaptive Boosting (AdaBoost) technique, a Random Forest algorithm, a neural network, a Support Vector Machine (SVM), a deep learning model, a convolutional neural network, a recurrent neural network, a transformer model.

18. A system for targeted monitoring of a patient in a bed comprising:
a depth sensing device configured to obtain an image of the bed;
a processing resource configured to:
receive the image of the bed;
predict, based on a posture of the patient in the image, a pressure score for one or more contact regions between the patient and the bed; and
provide an indication of the pressure score for the one or more contact regions.

19. The apparatus of claim 18, wherein the depth sensing device comprises at least one of: a depth sensing camera, a stereo camera, a camera cluster, a camera array, and a motion sensor.

20. A non-transitory machine-readable medium having instructions recorded thereon for execution by a processor to perform a set of operations, comprising:
receiving an image of a bed;
predicting, based on a posture of a patient in the image, a pressure score for one or more contact regions between the patient and the bed; and
providing an indication of the pressure score for the one or more contact regions.
